(19) United States
(12) Patent Application Publication
(10) Pub. No.: US 2019/0167816 A1
(43) Pub. Date: Jun. 6, 2019

(54) METHODS FOR PREVENTING OR TREATING FIBROTIC DISEASES

(71) Applicant: THE REGENTS OF THE UNIVERSITY OF CALIFORNIA, Oakland, CA (US)

(72) Inventors: Gino Cortopassi, Davis, CA (US); Alexey Tomilov, Sacramento, CA (US); Natalie Torok, Folsom, CA (US); XiaoSong Jiang, Davis, CA (US)

(73) Assignee: THE REGENTS OF THE UNIVERSITY OF CALIFORNIA, Oakland, CA (US)

(21) Appl. No.: 16/263,406

(22) Filed: Jan. 31, 2019

Related U.S. Application Data
(63) Continuation of application No. PCT/US2017/038591, filed on Jun. 21, 2017.
(60) Provisional application No. 62/369,616, filed on Aug. 1, 2016.

(51) Int. Cl. A61K 48/00; A61P 1/16; A61P 9/00; A61P 11/00

(52) U.S. Cl. CPC A61K 48/0066; A61P 1/16; A61K 48/0058; A61K 45/06; A61P 9/00; A61P 11/00; A61K 48/0008

(57) ABSTRACT

The present invention provides methods for preventing or treating a fibrotic disease in a subject. In some embodiments, the fibrotic disease is fatty liver disease, non-alcoholic fatty liver disease, or non-alcoholic steatohepatitis. In particular aspects, the methods comprise administering an inhibitor of Shc gene expression to achieve genetic suppression of Shc activity in the subject. In other aspects, the methods comprise administering a peptide, peptoid, or peptide-peptoid hybrid inhibitor of Shc to achieve pharmacological suppression of Shc protein activity in the subject.

### CROSS-REFERENCE TO RELATED APPLICATIONS

This application is a continuation of International Patent Application No. PCT/US2017/038591, filed Jun. 21, 2017, which claims priority to U.S. Provisional Application No. 62/369,616 filed Aug. 1, 2016, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with the Government support under Grant No. 5R01DK083283, awarded by the National Institutes of Health. The Government has certain rights in this invention.

### REFERENCE TO A “SEQUENCE LISTING”

The Sequence Listing written in file SequenceListing_070772-223610US-1123446.txt created on Jan. 30, 2019, 3,315 bytes, machine format IBM-PC, MS-Windows operating system, is hereby incorporated by reference in its entirety for all purposes.

### BACKGROUND OF THE INVENTION

The liver, a vital organ that is both the heaviest internal organ and the largest gland in the human body, is responsible for a wide range of critical functions, including metabolism, detoxification, protein synthesis, and the production of biochemicals that are necessary for digestion. The liver is responsible for the production of bile, which aids in digestion, plays important roles in carbohydrate metabolism (including glycogenesis and gluconeogenesis) and lipid metabolism (including cholesterol synthesis and lipogenesis), and produces blood clotting factors, among other functions. Also of note, the liver is a critical site for the metabolism of insulin and is crucial for the detoxification of many metabolites.

Fatty liver disease (FLD), also known simply as fatty liver or hepatic steatosis, is a condition wherein large vacuoles of triglyceride fat accumulate in hepatocytes via the process of steatosis (i.e., infiltration of liver cells with fat). While FLD can be caused by excessive alcohol consumption, people who drink little or no alcohol can also develop FLD, known as non-alcoholic fatty liver disease (NAFLD). The accumulation of fat in liver cells leads to inflammation and the development of fibrosis within the liver. As the extent of liver fibrosis increases, the development of more severe non-alcoholic steatoheptatitis (NASH) occurs. Accompanying the progression of liver fibrosis due to NAFLD and NASH is a progressive deterioration of liver function, eventually leading to liver failure.

NAFLD and NASH are severe, potentially life-threatening diseases, as reduction of liver function results in failures of glucose regulation, digestion, blood clotting, and the processing and detoxification of a large number of biochemicals, among other sequelae. NAFLD and NASH tend to develop in people who are obese and/or have diabetes, although NAFLD and NASH can develop in individuals without either of these risk factors. It is estimated that NAFLD affects 20-30% of people in Western countries (Bellentani, et al., Digestive Diseases, 28:155-61 (2010)).

As discussed above, NAFLD and NASH lead to progressive liver fibrosis and eventual organ failure. However, many other tissue and organ systems can also be effected by fibrotic diseases, often resulting from inflammation or other injury, with the potential for severe illness and/or loss of life. For example, fibrosis can affect the lungs (e.g., pulmonary fibrosis, cystic fibrosis), heart (e.g., atrial fibrosis, endomyocardial fibrosis, and fibrosis resulting from myocardial infarction), brain (e.g., glial scar), joints (e.g., arthrofibrosis of the knee, shoulder, or other joints), intestine (e.g., Crohn's Disease), skin (e.g., keloids, nephrogenic systemic fibrosis, scleroderma), bone marrow (e.g., myelofibrosis), penis (e.g., Peyronie's Disease), hands and fingers (e.g., Dupuytren's contracture), abdomen (e.g., retroperitoneal fibrosis), and chest cavity (e.g. mediastinal fibrosis).

For many of the fibrotic diseases discussed above, there are very few or no long-term effective treatments or cures. In some cases, the only available solutions carry significant risk and/or expense. For example, currently the only long-term solution for NAFLD and NASH is liver transplantation, which is risky, expensive, and requires constant and complicated immunosuppression. Accordingly, there is a need for new therapies for the prevention and treatment of fibrotic diseases. The present invention satisfies this need and provides related advantages as well.

### BRIEF SUMMARY OF THE INVENTION

In one aspect, the present invention provides a method for preventing or treating a fibrotic disease in a subject, the method comprising administering to the subject an effective amount of an inhibitor of Shc gene expression to achieve genetic suppression of Shc activity in the subject.

In some embodiments, the fibrotic disease is selected from the group consisting of fibrotic liver disease, pulmonary fibrosis, cardiac fibrosis, and cystic fibrosis. In some instances, the fibrotic disease is non-alcoholic fatty liver disease. In other instances, the fibrotic disease is non-alcoholic steatohepatitis.

In other embodiments, the inhibitor of Shc gene expression comprises DNA, RNA, a nuclease, or combinations thereof. In particular embodiments, the inhibitor of Shc gene expression is administered to perform RNA interference (RNAi), antisense therapy, CRISPR genome editing, a virus-mediated knockdown, or a combination thereof

In some other embodiments, the inhibitor of Shc gene expression is administered before the subject exhibits any symptoms of the fibrotic disease. In still other embodiments, the subject exhibits one or more symptoms of the fibrotic disease. In some instances, the administration of the inhibitor of Shc gene expression ameliorates at least one of the one or more symptoms.

In some other embodiments, the suppression of Shc activity in the subject is transient.

In particular embodiments, the level of one or more biomarkers indicative of the fibrotic disease is abnormal. In some instances, the one or more biomarkers indicative of the fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ). In other embodiments, the level of one or more biomarkers indicative of liver disease is abnormal. In some instances, the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin. In still other embodiments, the level of the one or more biomarkers is measured before administration of the inhibitor of Shc gene expression. In some instances, administration of the inhibitor of Shc gene expression results in the level of at least one of the one or more biomarkers returning to a control level.

In another aspect, the present invention provides a method for preventing or treating a fibrotic disease in a subject, the method comprising administering to the subject an effective amount of a peptide inhibitor of Shc, a peptoid inhibitor of Shc, a peptide-peptoid hybrid inhibitor of Shc, or a combination thereof to achieve pharmacological suppression of Shc protein activity in the subject.

In some embodiments, the fibrotic disease is selected from the group consisting of fibrotic liver disease, pulmonary fibrosis, cardiac fibrosis, and cystic fibrosis. In some instances, the fibrotic disease is non-alcoholic fatty liver disease.

In some embodiments, the peptide inhibitor, peptoid inhibitor, peptide-peptoid hybrid inhibitor, or combination thereof is administered before the subject exhibits any symptoms of the fibrotic disease. In still other embodiments, the subject exhibits one or more symptoms of the fibrotic disease. In some instances, the administration of the peptide inhibitor, peptoid inhibitor, peptide-peptoid hybrid inhibitor, or combination thereof ameliorates at least one of the one or more symptoms.

In particular embodiments, the level of one or more biomarkers indicative of the fibrotic disease is abnormal. In some instances, the one or more biomarkers indicative of the fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ). In other embodiments, the level of one or more biomarkers indicative of liver disease is abnormal. In some instances, the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin. In still other embodiments, the level of the one or more biomarkers is measured before administration of the peptide inhibitor, peptoid inhibitor, peptide-peptoid hybrid inhibitor, or combination thereof. In some instances, administration of the peptide inhibitor, peptoid inhibitor, peptide-peptoid hybrid inhibitor, or combination thereof results in the level of at least one of the one or more biomarkers returning to a control level.

Other objects, features, and advantages of the present invention will be apparent to one of skill in the art from the following detailed description and figures.

### BRIEF DESCRIPTION OF THE DRAWINGS

FIGS. 1A-1E show that Shc quantity and activity is increased in mice that are fed a “fatty liver diet”. FIG. 1A shows a Western blot illustrating p66Shc, p52Shc, and p46Shc Shc isoform expression in mice that were fed normal chow or the western diet (WD). FIG. 1B shows quantified p52Shc expression in mice that were fed normal chow or the WD. FIG. 1C shows quantified p46Shc expression in mice that were fed normal chow or the WD. FIG. 1D shows quantified phosphorylation of tyrosine residue 318 of p52Shc in mice that were fed normal chow or the WD. FIG. lE shows quantified phosphorylation of tyrosine residues 239 and 240 of p52Shc in mice that were fed normal chow or the WD.

FIGS. 2A and 2B show that genetic and pharmacological Shc inhibition suppress fat-induced toxicity in mouse hepatocytes. FIG. 2A shows apoptosis rates for hepatocytes from wild type control and Shc knock-out mice that were exposed to BSA or 100 μM palmitate (Pal) for 24 hours. FIG. 2B shows apoptosis rates for hepatocytes from wild type control mice that were exposed to BSA, 100 μM palmitate (Pal), or a combination of palmitate and the Shc inhibitor idebenone (10 μM) (Pal+Inh I) for 24 hours.

FIGS. 3A-3F show that genetic Shc inhibition ameliorates fatty liver disease in vivo after 19 weeks of NAFLD development. Mice were fed either normal chow or a western diet (WD) for 20 weeks. Twice during the 20^{th} week, mice that were fed the WD were injected with either a Shc knockdown construct (sh Shc) or a scrambled shRNA construct that is not specific for Shc (sh scr). Mice were sacrificed and tissues harvested at the end of the 20^{th} week. FIG. 3A shows the expression levels of alpha-smooth muscle actin (αSMA) in each of the three groups. FIG. 3B shows the expression levels of monocyte chemoattractant protein-1 (MCP1) in each of the three groups. FIG. 3C shows the expression levels of procollagen α1 (procol1) in each of the three groups. FIG. 3D shows the expression levels of interleukin-1β (IL-1b) in each of the three groups. FIG. 3E shows the expression levels of transforming growth factor-β (TGFβ) in each of the three groups. FIG. 3F shows the expression levels of tumor necrosis factor alpha (TNFα) in each of the three groups.

FIGS. 4A-4C show the chemical structures of some compounds that can be used as a starting point for the synthesis of peptide and peptide-peptoid hybrid inhibitors of Shc. FIG. 4A shows a peptide of the sequence Ac-pY-Q-G-L-S—NHR (SEQ ID NO:1), where “R” can be allyl or homoallyl (i.e., n=1 or 2). N-allyl substitution can occur at positions 1, 2, or 3 to yield peptide or peptide hybrid inhibitors of Shc. FIG. 4B shows a peptide of the sequence Ac-pY-Q-G-L—NHR (SEQ ID NO:2), where “R” can be ally! or homoallyl (i.e., n=1 or 2). N-allyl substitution can occur at positions 1, 2, or 3 to yield peptide or peptide hybrid inhibitors of Shc. FIG. 4C shows a peptide of the sequence Ac-pY-Q-G-L-amide (SEQ ID NO:3), where “R” can be substituted to yield different peptide-peptoid hybrid inhibitors of Shc.

### DETAILED DESCRIPTION OF THE INVENTION

### I. Introduction

Fatty liver diseases, including non-alcoholic fatty liver disease (NAFLD) and non-alcoholic steatohepatitis (NASH), are characterized by fat infiltration of hepatocytes, followed by inflammation resulting from fat toxicity. As in fibrotic diseases that affect many other tissues and organs throughout the body, the inflammation in NAFLD and NASH leads to the progressive development of fibrosis throughout the liver, which in turn leads to reduced liver function and eventually liver failure, with dire consequences for the affected individual. The present invention is based, in part, on the discovery that a fatty diet, known to induce fatty liver disease, causes an increase in the quantity and activity of Shc, as well as the surprising discovery that inhibition of Shc protects hepatocytes from fat toxicity and reverses the liver inflammation associated with fatty liver disease. Thus, in some embodiments, the invention provides methods for preventing or treating a fibrotic disease in a subject, the method comprising administering to the subject an effective amount of an inhibitor of Shc gene expression to achieve genetic suppression of Shc activity in the subject. In other embodiments, the invention provides methods for preventing or treating a fibrotic disease in a subject, the method comprising administering to the subject an effective amount of a peptide inhibitor of Shc, a peptoid inhibitor of Shc, a peptide-peptoid hybrid inhibitor of Shc, or a combination thereof to achieve pharmacological suppression of Shc protein activity in the subject. The methods of the present invention can not only protect a subject from the sequelae of fibrotic diseases such as fatty liver disease, they can also reverse the sequelae of such diseases. Thus, the present invention provides superior alternative solutions to fibrotic diseases for which, at least in some cases, the only current viable long-term solution is risky and expensive organ transplantation.

### II Definitions

As used herein, the following terms have the meanings ascribed to them unless specified otherwise.

An “effective amount” or “therapeutically effective amount” includes an amount or quantity effective, at dosages and for periods of time necessary, to achieve the desired therapeutic or prophylactic result.

The term “subject” typically includes humans, but can also include other animals such as, e.g., other primates, rodents, canines, felines, equines, ovines, porcines, and the like.

The terms “administering” and “administration” include oral administration, topical contact, administration as a suppository, intravenous, intraperitoneal, intramuscular, intralesional, intrathecal, intranasal (e.g., inhalation, nasal mist or drops), or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a Shc inhibitor of the invention for preventing or relieving one or more symptoms associated with the presence or activity of a fibrotic disease. By “co-administer” it is meant that a Shc inhibitor of the invention is administered at the same time, just prior to, or just after the administration of a second drug.

The terms “treating” and “treated” refer to any indications of success in the treatment or amelioration of a pathology or condition, including any objective or subjective parameter such as abatement, remission, diminishing of symptoms or making the pathology or condition more tolerable to the subject, slowing in the rate of degeneration or decline, making the final point of degeneration less debilitating, or improving a subject's physical or mental well-being. The treatment or amelioration of symptoms can be based on objective or subjective parameters, including the results of a physical examination, histopathological examination (e.g., analysis of biopsied tissue), laboratory analysis of urine, saliva, tissue sample (e.g., obtained from a biopsy), serum, plasma, or blood, or imaging.

The terms “antisense technology” and “antisense therapy” refer to a method of reducing or inhibiting the expression of a target gene wherein a nucleic acid (e.g., DNA, RNA, or an analog thereof) that is complementary to at least part of the mRNA of a target gene is introduced into a cell, thereby binding to the mRNA of the target gene and reducing or inhibiting expression of the target gene. Reduction or inhibition of expression can occur either as a consequence of steric interference, thereby preventing translation of the mRNA of the target gene, or recruitment of RNase H, which degrades the mRNA of the target gene and prevents translation, or a combination thereof

The term “interfering RNA” or “RNAi” or “interfering RNA sequence” as used herein includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. As used herein, the term “siRNA” includes RNA-RNA duplexes as well as DNA-RNA hybrids (see, e.g., PCT Publication No. WO 2004/078941).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript.

As used herein, the term “mismatch motif” or “mismatch region” refers to a portion of an interfering RNA sequence that does not have 100% complementarity to its target sequence. An interfering RNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The phrases “genetic suppression” and “inhibiting expression” mean to silence, reduce, or inhibit the expression of a target gene (e.g., Shc), and can refer to silencing, reducing, or inhibiting transcription, translation, or both. Genetic suppression can be either a partial or complete reduction or inhibition of the expression of the target gene. Also, genetic suppression can be transient or permanent. Non-limiting examples of genetic inhibitors include DNA, RNA, single-stranded nucleic acids, double-stranded nucleic acids, nucleases, and combinations thereof. As non-limiting examples, genetic suppression can be accomplished using RNAi, antisense technology, CRISPR genome editing, CRISPR interference (i.e., a variant of CRISPR genome editing wherein the Cas9 enzyme lacks endonuclease activity and sterically interferes with transcription by blocking transcriptional initiation or elongation), virus-mediated knock-down, or combinations thereof. Additional methods of performing genetic suppression will be known to those of ordinary skill in the art. As a non-limiting example of how to examine the extent of gene silencing, a test sample (e.g., a sample of cells in culture expressing the target gene) or a test mammal (e.g., a mammal such as a human or an animal model such as a rodent (e.g., mouse) or a non-human primate (e.g., monkey) model) is contacted with an interfering RNA that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal model such as a rodent (e.g., mouse) or non-human primate (e.g., monkey) model) that is not contacted with or administered the interfering RNA. The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the genetic suppression agent is capable of silencing, reducing, or inhibiting the expression of a target gene (e.g., Shc) by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the genetic suppression agent. Suitable assays for determining the level of target gene expression include, without limitation, examination of protein or mRNA levels using techniques known to those of skill in the art, such as, e.g., dot blots, Northern blots, in situ hybridization, ELISA, immunoprecipitation, Western blots, enzyme function, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed- base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide (e.g., Shc).

The term “Shc” refers to adapter proteins of the SH2 domain family that are encoded by the SHC1 gene. Shc is also known as SHC1, SHC, SHCA, SHC adaptor protein 1, SHC-transforming protein 1, SHC (Src Homology 2 Domain Containing) Transforming Protein 1, SHC (Src Homology 2 Domain-Containing) Transforming Protein 1, SHC-Transforming Protein 3, SHC-Transforming Protein A, SH2 Domain Protein C1, and Src-homology-collagen protein. Non-limiting examples of Shc amino acid sequences are set forth in GenBank Accession Nos. NM_183001.4 4→NP_892113.4 (human isoform 1), NM_003029.4 4→NP_003020.2 (human isoform 2), NM_001130040.1→NP_001123512.1 (human isoform 3), NM_001130041.1→NP_001123513.1 (human isoform 4), NM_001202859.1 →NP_001189788.1 (human isoform 5 precursor), NM_001113331.2 →NP_001106802.1 (mouse isoform a), and NM_011368.5→NP_035498.2 (mouse isoform b). There are three main Shc protein isoforms, each differing in activity and subcellular location: p46Shc, p52Shc, and p66Shc (having molecular weights of 46 kDa, 52 kDa, and 66 kDa, respectively). All three proteins have an N-terminal phosphotyrosine-binding (PTB) domain, a central collagen homology domain (CH1 domain), and a C-terminal Src-homology 2 (SH2) domain. The PTB and SH2 domains for all three isoforms can bind to tyrosine-phosphorylated proteins, but they have different phosphopeptide-binding specificities. The p66Shc isoform also has an additional N-terminal collagen homology domain (CH2 domain). The p46Shc and p52Shc isoforms activate the Ras pathway by recruiting the GRB2/SOS complex. On the other hand, p66Shc is not involved with Ras-ERK activation. The p46Shc isoform, unlike the p52Shc and p66Shc isoforms, is targeted to the mitochondrial matrix. Acting downstream of the tumor suppressor p53, p66Shc plays a role in elevating intracellular oxidants, the release of cytochrome c, and apoptosis. Shc has been shown to transmit signals of cell surface receptors such as EGFR, erbV-2, and insulin receptors, and increased Shc activity is associated with cancer cell mitogenic activity and metastasis.

The term “pharmacological inhibition of Shc protein activity” means to reduce or inhibit the ability of a Shc protein to perform any of its normal functions, effected, for example, by a peptide inhibitor, a peptoid inhibitor, a peptide-peptoid hybrid inhibitor, or a combination thereof. Non-limiting examples of inhibition of Shc protein activity include inhibiting the ability of Shc to interact with a binding partner at the Shc PTB domain (e.g., inhibiting the ability of an insulin receptor to bind to the Shc PTB domain), inhibiting the ability of one or more Shc tyrosine residues to be phosphorylated (e.g., inhibiting or blocking phosphorylation of tyrosine residues 239, 240, 317, and/or 318), inhibiting the ability of Shc to interact with a binding partner at the Shc CH1 domain (e.g., inhibiting the ability of Grb2 to bind to the Shc CH1 domain), and inhibiting the ability of Shc to interact with a binding partner at the Shc SH2 domain (e.g., inhibiting the ability of EGFR to bind to the Shc SH2 domain).

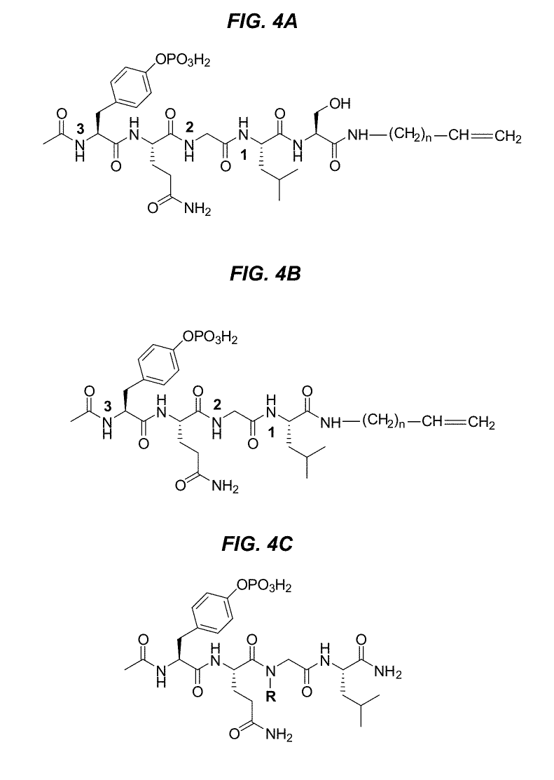

The term “peptide inhibitor” means a peptide that acts to inhibit or suppress a biological process. As a non-limiting example, a peptide inhibitor may inhibit Shc protein activity by interfering with binding at the PTB, CH1, CH2, or SH2 domains of Shc, or a combination thereof. As another non-limiting example, a peptide inhibitor may inhibit phosphorylation of one or more Shc tyrosine residues. Examples of peptide inhibitors that interfere with binding of the Shc SH2 domain include, without limitation, peptides comprising the sequences pY-Q-G-L-S-amide (SEQ ID NO:4), NH—(CH₂)₂—CO-pY-Q-G-L-S-amide (SEQ ID NO:5), NH—(CH₂)₃—CO-pY-Q-G-L-S-amide (SEQ ID NO:6), NH—(CH₂)₄-CO-pY-Q-G-L-S-amide (SEQ ID NO:7), NH—(CH₂)₅—CO-pY-Q-G-L-S-amide (SEQ ID NO:8), NH—(CH₂)₂—CO-L-pY-Q-G-L-S-amide (SEQ ID NO:9), NH—(CH₂)₃—CO-L-pY-Q-G-L-S-amide (SEQ ID NO:10), and NH—(CH₂)₄—CO-L-pY-Q-G-L-S-amide (SEQ ID NO:11), where “pY” represents phosphorylated tyrosine, and “L,” “Q,” “G,” and “S” are standard one-letter amino acid symbols. Further non-limiting examples of peptide inhibitors that interfere with Shc SH2 domain binding are illustrated in FIGS. 4A and 4B. Additional non-limiting examples of peptide inhibitors, including peptides predicated on the sequence X-pY-Q-G-L-S-amide (SEQ ID NO:12), where “X” represents a variety of spacer groups, are disclosed in Choi, et al., J. Med. Chem., 52:1612-1618 (2009); incorporated herein by reference in its entirety for all purposes.

The term “peptoid inhibitor” means a peptoid that acts to inhibit or suppress a biological process. As a non-limiting example, a peptoid inhibitor may inhibit Shc protein activity by interfering with binding at the PTB, CH1, CH2, or SH2 domains of Shc, or a combination thereof. As another non-limiting example, a peptoid inhibitor may inhibit phosphorylation of one or more Shc tyrosine residues.

The term “peptide-peptoid hybrid inhibitor” means a peptide-peptoid hybrid that acts to inhibit or suppress a biological process. As a non-limiting example, a peptide-peptoid hybrid inhibitor may inhibit Shc protein activity by interfering with binding at the PTB, CH1, CH2, or SH2 domains of Shc, or a combination thereof. As another non-limiting example, a peptide-peptoid hybrid inhibitor may inhibit phosphorylation of one or more Shc tyrosine residues. Examples of peptide-peptoid hybrid inhibitors that interfere with binding of the Shc SH2 domain include, without limitation, the compounds that result when the compounds depicted in FIGS. 4A and 4B are N-allyl substituted at the nitrogen atom depicted as position #2. Additional non-limiting examples of peptide-peptoid hybrid inhibitors that inhibit Shc SH2 domain binding include a variety of compounds predicated on the structure shown in FIG. 4C. The “R” group can be any group, including but not limited to ethyl, n-propyl, n-butyl, n-pentyl, n-hexyl, 3-methylbutyl, 4-methylpentyl, 4-fluorobutyl, 4,4,4-trifluorobutyl, 2-(methoxy)ethyl, 2-(ethoxy)ethyl, 2-(ethylthio)ethyl, allyl, but-3-en-1-yl, pent-4-en-1-yl, hex-5-en-1-yl, but-2-yn-1-yl, pent-2-yn-1-yl, and benzyl groups. Additional non-limiting examples of peptide-peptoid hybrid inhibitors are disclosed in Choi, et al., J. Med. Chem., 52:1612-1618 (2009); incorporated herein by reference in its entirety for all purposes.

The terms “polypeptide,” “peptide,” and “protein” are used interchangeably herein to refer to a polymer of amino acid residues, or an assembly of multiple polymers of amino acid residues.

The term “amino acid” includes but is not limited to naturally-occurring a-amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring amino acids are those encoded by the genetic code, as well as those amino acids that are later modified (e.g., hydroxyproline, y-carboxyglutamate, and O-phosphoserine). Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The term “peptoid” refers to a polyamide of between about 2 and 100 (e.g., between about 2 and 90, 2 and 80, 2 and 70, 2 and 60, 2 and 50, 2 and 40, 2 and 30, 2 and 20, 2 and 10, 2 and 9, 2 and 8, 2 and 7, 2 and 6, 2 and 5, 2 and 4, or 2 and 3) units having substituents “R” on the amide nitrogen atoms. A peptoid is a synthetic analog of a peptide with the difference being that while a side-chain residue on a peptide is attached to a carbon atom that is a- to the carbonyl group, in a peptoid, the “side-chain residue” is attached to the amide nitrogen atom. Peptoids are synthetic polymers with controlled sequences and lengths, that can be made by automated solid-phase organic synthesis to include a wide variety of side-chains having different chemical functions. “R” groups bonded to the amide nitrogen atoms in the peptoids can include, but are not limited to, straight-chain and branched alkyl groups (e.g., ethyl, n-propyl, n-butyl, n-pentyl, n-hexyl, 3-methylbutyl, 4-methylpentyl, and the like); haloalkyl groups (e.g., 4-fluorobutyl, 4,4,4-trifluorobutyl, and the like); heteroalkyl (e.g., 2-(methoxy)ethyl, 2-(ethoxy)ethyl, 2-(ethylthio)ethyl, and the like); alkenyl (e.g., allyl, but-3-en-1-yl, pent-4-en-1-yl, hex-5-en-1-yl, and the like); alkynyl (e.g., but-2-yn-1-yl, pent-2-yn-1-yl, and the like); and arylalkyl groups (e.g., benzyl and the like). In some embodiments, peptoid “R” groups are alkyl groups wherein each alkyl group is optionally and independently selected from the group consisting of halo, hydroxy, amino, alkylamino, amido, acyl, nitro, cyano, and alkoxy.

The term “peptide-peptoid hybrid” refers to an oligomer that is composed of both alpha amino acids and N-substituted glycine residues (i.e., an oligomer that is composed of peptide and peptoid units).

The term “alkyl,” by itself or as part of another substituent, refers to a straight or branched, saturated, aliphatic radical having the number of carbon atoms indicated. Alkyl can include any number of carbons, such as C₁₋₂, C_{1-b 3}, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, alkylamino, amido, acyl, nitro, cyano, and alkoxy.

The term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. “Substituted alkenyl” groups can be substituted with one or more groups selected from halo, hydroxy, amino, alkylamino, amido, acyl, nitro, cyano, and alkoxy.

The term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted. “Substituted alkynyl” groups can be substituted with one or more groups selected from halo, hydroxy, amino, alkylamino, amido, acyl, nitro, cyano, and alkoxy.

The term “heteroalkyl,” by itself or as part of another substituent, refers to an alkyl group of any suitable length and having from 1 to 3 heteroatoms such as N, O and S. For example, heteroalkyl can include ethers, thioethers and alkyl-amines. Additional heteroatoms including, but not limited to, B, Al, Si and P, can also be useful. The heteroatoms can be oxidized to form moieties such as —S(O)— and —S(O)₂—. The heteroatom portion of the heteroalkyl can replace a hydrogen of the alkyl group to form a hydroxy, thio, or amino group. Alternatively, the heteroatom portion can be the connecting atom, or be inserted between two carbon atoms.

The term “aryl,” by itself or as part of another substituent, refers to an aromatic ring system having any suitable number of ring atoms and any suitable number of rings. Aryl groups can include any suitable number of ring atoms, such as 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. “Substituted aryl” groups can be substituted with one or more groups selected from halo, hydroxy, amino, alkylamino, amido, acyl, nitro, cyano, and alkoxy.

The term “alkoxy,” by itself or as part of another substituent, refers to a group having the formula —OR, wherein R is alkyl.

The terms “halo” and “halogen,” by themselves or as part of another substituent, refer to a fluorine, chlorine, bromine, or iodine atom.

The term “amino” refers to a moiety —NR₃, wherein each R group is H or alkyl. An amino moiety can be ionized to form the corresponding ammonium cation.

The term “hydroxy” refers to the moiety —OH.

The term “cyano” refers to a carbon atom triple-bonded to a nitrogen atom (i.e., the moiety —C≡N).

The term “carboxy” refers to the moiety —C(O)OH. A carboxy moiety can be ionized to form the corresponding carboxylate anion.

The term “amido” refers to a moiety —NRC(O)R or —C(O)NR₂, wherein each R group is H or alkyl.

The term “isoform” means a variant of a protein, peptide, nucleic acid, or other biological chemical. As a non-limiting example, different mRNA isoforms (i.e., mRNAs of different lengths and/or containing different nucleotide sequences) can be transcribed from the same region of genomic DNA. As another non-limiting example, there are three main Shc isoforms (i.e., three main variants of the Shc protein), each having a different size.

The term “nuclease” means an enzyme that is capable of cleaving the phosphodiester bonds between the nucleotide subunits of a nucleic acid. An “endonuclease” is a nuclease that cleaves phosphodiester bonds within a nucleic acid. Endonucleases can be specific (i.e., cleaving phosphodiester bonds within specific nucleotide sequences), or non-specific (i.e., cleaving phosphodiester bonds without regard to the flanking nucleotide sequences).

The term “virus-mediated knockdown” refers to the use of a viral vector to deliver a nucleic acid (e.g., an interfering RNA) to a target cell or tissue for the purpose of achieving genetic suppression of one or more target genes. As non-limiting examples, lentivirus and adeno-associated virus systems can be used to deliver interfering RNA in order to suppress gene expression by RNAi. Delivery can be systemic or targeted to specific tissues, depending on the choice of viral delivery system.

The terms “CRISPR genome editing” and “CRISPR” refer to a technique for genome editing and regulation that employ clustered, regularly interspaced, short palindromic repeat (CRISPR) sequences. By delivering the appropriate guide RNAs and a nuclease (e.g., Cas9 or Cpf1) to a cell, the cell's genome can be cut and genetic sequences deleted or added at a specified location. CRISPRs have been used with specific endonuclease enzymes for genome editing and regulation throughout a large variety of organisms. CRISPR interference (CRISPRi) is a variant of CRISPR wherein the Cas9 enzyme lacks endonuclease activity and sterically interferes with transcription by blocking transcriptional initiation or elongation. For more information about CRISPR, see, e.g., Sander, et al., Nature Biotechnology, 32:347-355 (2014); incorporated herein by reference in its entirety for all purposes.

The term “alpha-smooth muscle actin or “αSMA” refers to a protein that is encoded by the ACTA2 gene, and is commonly used as a marker of myofibroblast formation. αSMA belongs to the actin family of proteins, which are highly conserved and play roles in cell motility, structure, and integrity. The gene encoding aSMA is also known as AAT6, ACTSA, or MYMY5, and is located on human chromosome 10. Non-limiting examples of αSMA amino acid sequences are set forth in GenBank Accession Nos. NM_001141945.2→NP_001135417.1 (human), NM_001613.2→NP_001604.1 (human), and NM_007392.3→NP_031418.1 (mouse).

The term “monocyte chemoattractant protein-1” or “MCP1” refers to a small cytokine that belongs to the CC chemokine family and is encoded by the CCL2 gene. MCP1 is also known as chemokine (C-C motif) ligand 2 (CCL2) or small inducible cytokine A2. The CCL2 gene is found on chromosome 17 in humans. MCP1 recruits monocytes, memory T cells, and dendritic cells to sites of inflammation. Non-limiting examples of MCP1 amino acid sequences are set forth in GenBank Accession Nos. NM_002982.3→NP_002973.1 (human) and NM_011331.2→NP_035461.2 (mouse).

The term “procollagen α1” or “procol1” refers to a protein encoded by the COL1A1 gene. A chain of procollagen α1 combines with a second chain of procollagen al and one chain of procollagen α2 (encoded by the COL1A2 gene) to form type I procollagen, which is then processed into type I collagen. Type I collagen is a fibrillary collagen found in most connective tissues in the body, including cartilage. Procol1 expression is associated with the development of fibrosis. Non-limiting examples of procoll amino acid sequences are set forth in GenBank Accession Nos. NM_000088.3→NP_000079.2 (human) and NM_007742.4→NP_031768.2 (mouse).

The term “interleukin-1β” or “IL-1b” refers to a cytokine that is also known as leukocytic pyrogen, mononuclear cell factor, lymphocyte activating factor, IL1B, IL-1, IL1-BETA, or IL1F2, and is encoded by the IL1B gene. IL-1b plays roles in the inflammatory response and is involved in cell proliferation, differentiation, and apoptosis, as well as the development of fibrosis.

The term “transforming growth factor-β ” or “TGFβ” refers to a multifunctional cytokine of the transforming growth factor superfamily that includes three different isoforms (TGFβ-1, TGFβ-2, and TGFβ-3). The three isoforms are encoded by the genes TGFB1, TGFB2, and TGFB3, respectively. TGFβ plays roles in cell proliferation, wound healing, and synthesis of extracellular matrix molecules. TGFβ is associated with the development of fibrosis in many different organs via its promotion of mesenchymal cell proliferation, migration, and accumulation following an inflammatory response.

The term “tumor necrosis factor alpha” or “TNFα” refers to the cytokine encoded by the gene TNF (also known as TNFA). TNFα is also known as tumor necrosis factor, TNF, TNFA, DIF, TNFSF2, cachexin or cachectin. TNFα is involved in systemic inflammation and is one of the cytokines that comprise the acute phase reaction. TNFα is produced primarily by activated macrophages, but is also produced by CD4+ lymphocytes, natural killer cells, neutrophils, mast cells, eosinophils, and neurons. Non-limiting examples of TNFα amino acid sequences are set forth in GenBank Accession Nos. NM_000594.3→NP_000585.2 (human) and NM_001278601.1→NP_001265530.1 (mouse).

The term “connective tissue growth factor” or “CTGF” refers to the matricellular protein of the CCN family of extracellular matrix-associated heparin binding proteins that is encoded by the CTGF gene. CTGF is also known as CCN2, HCS24, IGFBP8, or NOV2. Non-limiting examples of CTGF amino acid sequences are set forth in GenBank Accession Nos. NM_001901.2→NP_001892.1 (human) and NM_010217.2→NP_034347.2 (mouse). CTGF is associated with virtually all fibrotic pathology, in addition to wound healing. It has also been shown that CTGF cooperates with TGFβ to promote sustained fibrosis.

The term “platelet derived growth factor receptor beta” or “PDGFRβ” refers to the beta form of the platelet derived growth factor receptor that is encoded by the gene PDGFRB. Platelet derived growth factor receptor beta is also known as PDGFRB, CD140B, IBGC4, IMF1, JTK12, PDGFR, PDGFR-1, PDGFR1, KOGS, or PENTT. PDGFRβ is a cell surface tyrosine kinase receptor that, when activated following binding of a PDGF ligand and subsequent dimerization with another PDGFR beta receptor or a PDGFR alpha receptor, activates cellular signaling pathways that play roles in cell proliferation, differentiation, and growth. A non-limiting example of a PDGFRβ amino acid sequence is set forth in GenBank Accession No. NM_002609.3→NP_002600.1. PDGFRβ activation is associated with the replication, survival and migration of myofibroblasts during the progression of fibrotic diseases.

The term “aspartate aminotransferase” or “AST” refers to a pyridoxal phosphate (PLP)-dependent transaminase enzyme (Enzyme Commission number 2.6.1.1) that is also known as aspartate transaminase, AspAT, ASA, AAT, or serum glutamic oxaloacetic transaminase (SGOT). AST plays important roles in amino acid metabolism, catalyzing the transfer of alpha amino groups between aspartate and glutamate. AST is a common biochemical marker of liver disease, as it is released from liver cells following liver injury, manifesting as elevated AST concentrations when measured using a blood test. Normal AST reference ranges for blood tests are 8-40 IU/L for males and 6-34 IU/L for females. The ratio of AST to ALT is also a common clinical biomarker for liver disease.

The term “alanine aminotransferase” or “ALT” refers to a transaminase enzyme (Enzyme Commission number 2.6.1.2) that is also known as alanine transaminase, serum glutamate-pyruvate transaminase (SGPT), or serum glutamic-pyruvate transaminase (SGPT). ALT catalyzes the transfer of an amino group from L-alanine to a-ketoglutarate and plays important roles in the alanine cycle. ALT is a common biochemical marker of liver disease, as it is released from liver cells following liver injury, manifesting as elevated ALT concentrations when measured using a blood test. Normal ALT reference ranges for blood tests are ≤52 IU/L for males and ≥34 IU/L for females. The ratio of AST to ALT is also a common clinical biomarker for liver disease.

The term “AST to platelet ratio index” or “APRI” refers to a method of using a subject's AST level, as measured using a blood test, and the subject's platelet count to predict the amount of liver fibrosis in the subject, as non-invasive alternative to liver biopsy. APRI is calculated using the following formula:

$$APRI = \frac{\frac{AST level}{AST upper limit of normal}}{platelet count} \times 100$$

wherein the AST level and the AST upper limit of normal are expressed in units of IU/L and platelet count is expressed in units of 10⁹/L. A commonly-recommended value of AST upper limit of normal is 40 IU/L. Higher APRI values are associated with greater positive predictive values of liver fibrosis.

The term “gamma-glutamyl transferase” or “GGT” refers to an enzyme that transfers gamma-glutamyl functional groups and is also known as gamma-glutamyl transpeptidase, GGTP or gamma-GT (Enzyme Commission number 2.3.2.2). GGT catalyzes the transfer of the gamma-glutamyl moiety of glutathione to acceptors that include amino acids, peptides, and water (i.e., the formation of glutamate), and plays a role in the gamma-glutamyl cycle, which functions in glutathione degradation and drug detoxification. GGT is useful for determining whether an increase in alkaline phosphatase is due to skeletal disease (in which case GGT levels will be normal) or liver disease (in which case GGT will be elevated).

The terms “alkaline phosphatase,” “AP,” and “ALP” refer to the hydrolase enzyme (Enzyme Commission number 3.1.3.1) that is also known as alkaline phosphomonoesterase, phosphomonoesterase, glycerophosphatase, alkaline phosphohydrolase, alkaline phenyl phosphatase, or orthophosphoric-monoester phosphohydrolase (alkaline optimum). AP removes phosphate groups from many different molecules, including nucleotides, proteins, and alkaloids. When liver cells are damaged, AP is released, thus elevated levels of AP in blood tests can be indicative of liver disease.

The term “bilirubin” refers to the yellow breakdown product of normal heme catabolism, and has a chemical formula of C₃₃H₃₆N₄O₆ and a molar mass of 584.67 g/mol. Measurement of bilirubin can be “indirect” (i.e., unconjugated bilirubin) or “direct” (i.e., conjugated bilirubin). Normal bilirubin levels, when measured using a blood test, range between 0 and 0.3 mg/dl for conjugated bilirubin, and 0.3 to 1.9 mg/dl for total bilirubin (i.e., conjugated and unconjugated bilirubin combined). Bilirubin is excreted from the liver into the bile duct and stored in the gallbladder, and is released into the small intestine as bile to aid digestion. When liver function is impaired, bilirubin is not adequately removed from the blood, resulting in elevated bilirubin levels.

The term “ferritin” refers to a hollow globular protein having a molecular weight of 450 kDa and consisting of 24 subunits that functions to store iron in a non-toxic form and transport and release iron to areas where iron is needed. The light type ferritin subunit is encoded by the FTL gene, and the heavy type subunit is encoded by FTH1 gene (also known as FTHL6). Non-limiting examples of ferritin amino acid sequences are set forth in GenBank Accession Nos. NM_000146.3→NP_000137.2 (light chain) and NM_002032.2→NP_002023.2 (heavy chain). Ferritin is stored in many types of cells, including liver cells. When liver cells are damaged, ferritin is released, resulting in elevated serum ferritin levels. Serum ferritin levels greater than 300 ng/mL in men and 200 ng/mL in women are commonly considered to be abnormal.

The term “magnetic resonance elastography” or “MRE” refers to a technique which uses magnetic resonance imaging to obtain information about the stiffness of a tissue by measuring the propagation of mechanical waves through the tissue. MRE involves generating shear waves in the tissue of interest, followed by acquiring magnetic resonance images that depict the propagation of the induced shear waves through the tissue and analysis of the images to generate quantitative maps of tissue stiffness (i.e., elastograms). The mechanical stress that is used to generate the shear waves can be internal (e.g., respiration motion or cardiac pulsations) or can be generated by external mechanical sources. Non-limiting examples of tissues for which MRE is useful in assessing fibrosis are the liver and kidneys. For more information on MRE, see e.g., Mariappan et al., Clin. Anat., 23:497-511 (2010); incorporated herein by reference in its entirety for all purposes.

### III. Fibrotic Diseases

The methods of the present invention are useful for the prevention and treatment of a wide variety of fibrotic diseases. Fibrosis is the formation or deposition of an abnormal excess of fibrous connective tissue within an organ or tissue, often in response to inflammation or other injury. Fibrosis, which is similar to the process of scarring, involves connective tissue being laid down by fibroblasts that have been stimulated by biochemicals released by immune cells such as macrophages. Some of the biochemicals associated with fibroblast stimulation include TGFβ, which is released by macrophages and damaged tissues, CTGF, platelet derived growth factor (PDGF), and interleukin 4 (IL-4). The aforementioned signaling molecules activate signaling pathways such as AKT/mTOR and SMAD, with an ultimate increase in the proliferation and activation of fibroblasts. The activated fibroblasts then deposit extracellular matrix (ECM) into the surrounding connective tissue. These processes are beneficial in that they are necessary for tissue healing, and require tight balancing of ECM synthesis and degradation in order for normal tissue architecture and function to be maintained. However, especially in scenarios where tissue injury or insult is severe or repetitive, the fibrotic response can become pathological, leading to excessive fibrosis and disruption of normal tissue and organ structure and function.

Fibrosis can occur in many tissues and organ systems, including but not limited to the liver, lungs, heart, brain, joints, skin and connective tissues, bone marrow, and chest and abdominal cavities. Fibrotic diseases can be diagnosed and monitored by a variety of methods, including imaging (e.g., magnetic resonance imaging (MRI), magnetic resonance elastography (MRE), computed tomography (CT), and ultrasonography), and the measurement of various biomarkers. Biomarkers useful for the detection and monitoring of fibrotic diseases include but are not limited to alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ).

Fibrotic liver disease (which can progress to the development of liver cirrhosis) can be caused by a large number of diseases and drugs, including but not limited to fatty liver disease (e.g., NAFLD and NASH), autoimmune hepatitis, viral infection (e.g., hepatitis B or C), bacterial infection (e.g., brucellosis), parasitic infection (e.g., echinococcosis), primary biliary cirrhosis, primary sclerosing cholangitis, congenital hepatic cirrhosis, storage or metabolic diseases (e.g., alpha-1 antitrypsin deficiency, copper storage diseases, fructosemia, galactosemia, glycogen storage diseases, iron-overload diseases, lipid dysfunction, peroxisomal disorders, and tyrosinemia), Budd-Chiari syndrome, heart failure, hepatic veno-occlusive disease, portal vein thrombosis, amiodarone, alcohol, chlorpromazine, isoniazid, methotrexate, methyldopa, oxyphenisatin, and tolbutamide. Liver fibrosis can severely impair liver function, often with severe and potentially life-threatening effects, including but not limited to abnormal blood clotting, insufficient metabolism and detoxification of drugs, metabolites and other biochemical compounds, deficiencies of metabolism (including glucose, lipid and protein metabolism), ascites, and secondary damage to other organs.

Fatty liver disease (FLD), also known simply as fatty liver or hepatic steatosis, is a condition wherein large vacuoles of triglyceride fat accumulate in hepatocytes via the process of steatosis (i.e., infiltration of liver cells with fat). FLD can be caused by excessive alcohol consumption. However, FLD also occurs in individuals who consume little or no alcohol, in which case the disease is known as non-alcoholic fatty liver disease (NAFLD). The accumulation of fat in the liver leads to inflammation and the development of fibrosis within the liver. As the extent of liver fibrosis increases, the development of more severe non-alcoholic steatoheptatitis (NASH) occurs. Accompanying the progression of liver fibrosis due to NAFLD and NASH is a progressive deterioration of liver function, possibly leading to liver failure. FLD is estimated to affect about 10 to 20 percent of Americans, with an additional about 2 to 5 percent being affected by the more severe NASH. NASH is often first suspected in an individual who is found to have elevated levels of one or more biomarkers of liver disease (e.g., ALT and AST), particularly when there is no other apparent reason for liver disease (e.g., heavy alcohol intake, medication, or infection such as hepatitis). A suspicion of NASH may also occur when X-ray or other imaging studies show evidence of fatty liver. The gold standard for distinguishing NASH from more benign FLD is to perform a liver biopsy. Suitable biomarkers for the detection and monitoring of liver disease, including NAFLD and NASH, include but are not limited to aspartate aminotransferase (AST), alanine aminotransferase (ALT), the ratio of AST to ALT (i.e., the AST/ALT ratio is often greater than 2 in progressive NASH), gamma-glutamyl transferase (GGT), the aspartate to platelet ratio index (APRI), alkaline phosphatase (AP), bilirubin, and ferritin.

Pulmonary fibrosis is a condition that occurs when excessive fibrotic tissue develops in the lungs, leading to thickening and loss of elasticity, narrowing of structures, and impaired respiratory function. Symptoms of pulmonary fibrosis include but are not limited to shortness of breath, dry cough, and fatigue. Pulmonary fibrosis can be caused by various medical conditions (e.g., tuberculosis, pneumonia, systemic lupus erythematosus, rheumatoid arthritis, sarcoidosis, scleroderma, and cystic fibrosis), tobacco smoking, occupational and environmental exposure (e.g., silica dust, asbestos fibers, grain dust, coal dust (e.g., progressive massive fibrosis), and bird and other animal droppings), radiation treatments, chemotherapy drugs (e.g., methotrexate, cyclophosphamide), cardiac medications (e.g., amiodarone, propranolol), and antibiotics (e.g., nitrofurantoin, sulfasalazine). In many cases, the cause of pulmonary fibrosis in an individual is idiopathic (i.e., unknown).

Cystic fibrosis is a genetic disorder that affects the lungs, pancreas, liver, kidneys, and intestine. Cystic fibrosis is caused by defective cystic fibrosis transmembrane conductance regulator (CFTR) protein. CFTR plays a role in the production of secretions such as sweat, digestive fluids, and mucus, and the defective CFTR leads to secretions that are abnormally thick and non-functional. This can lead to the accumulation of secretions in the affected organs, causing injury and the development of fibrosis. Cystic fibrosis patients are often afflicted with frequent lung infections, coughing up of mucus, and eventual respiratory failure. Narrowing of structures and fibrosis in other organs such as the pancreas and liver can lead to reduced functionality of these organs as well.

Cardiac fibrosis can result from the inappropriate proliferation of fibroblasts and excessive deposition of fibrous tissue either in the cardiac muscle or within or on the heart valves. Fibrosis of the cardiac muscle can lead to stiffening and decreased pumping ability of the heart (e.g., decreased ejection fraction) or abnormal electrical functioning (e.g., increased risk of lethal cardiac arrhythmia). Fibrosis can occur in any chamber (e.g., atrial fibrosis, ventricular fibrosis) and can affect various layers of the myocardium (e.g., endomyocardial fibrosis). Fibrosis can also occur as the result of a previous myocardial infarction. Fibrosis of the cardiac valves leads to thickening of the valves. Often the tricuspid valve is affected, but the pulmonary and other valves can be affected as well. Thickening of the valves can lead to valvular dysfunction and eventually heart failure.

In addition to the fibrotic diseases disclosed above and elsewhere herein, other non-limiting examples of fibrotic diseases for which the methods of the present invention are useful include scleroderma (an autoimmune disease of the connective tissues that primarily affects the skin, but can also involve other organs such as the kidneys, lungs, and heart), as well as fibrosis of the brain (e.g., glial scar), joints (e.g., arthrofibrosis of the knee, shoulder, or other joints, adhesive capsulitis of the shoulder), intestine (e.g., Crohn's Disease), skin (e.g., keloids, nephrogenic systemic fibrosis), bone marrow (e.g., myelofibrosis), penis (e.g., Peyronie's Disease), hands or fingers (e.g., Dupuytren's contracture), abdomen (e.g., retroperitoneal fibrosis (including the aorta, kidney, and surrounding structures)), and chest cavity (e.g. mediastinal fibrosis). Mediastinal fibrosis is often characterized by calcified fibrosis of the lymph nodes, which can lead to blockage of respiratory structures and blood vessels. Bone marrow fibrosis can result in impaired production of blood cells in the bone marrow. Fibrosis of the joints can cause pain, stiffening, or impaired mobility or range of motion of the shoulders, knees, wrists, and hips, and other joints, while Dupuytren's contracture can result in deformity of the hands and fingers.

### IV. Detailed Description of the Embodiments

In some aspects, the methods of the present invention for preventing or treating a fibrotic disease in a subject comprise administering to the subject an effective amount of an inhibitor of Shc gene expression to achieve genetic suppression of Shc activity in the subject.

In some embodiments, the fibrotic disease is selected from the group consisting of fibrotic liver disease, pulmonary fibrosis, cardiac fibrosis, and cystic fibrosis. In particular embodiments, the fibrotic disease is a fatty liver disease. In some instances, the fatty liver disease is non-alcoholic fatty liver disease (NAFLD). In other instances, the fatty liver disease is non-alcoholic steatohepatitis. In yet other embodiments, the subject has a plurality of fibrotic diseases or is at risk for developing one or more fibrotic diseases. In some instances, the subject has diabetes or has one or more risk factors for developing diabetes. In other instances, the subject is obese or has one or more risk factors for becoming obese.

In some embodiments, the inhibitor of Shc gene expression comprises DNA, RNA, a nuclease, or combinations thereof. In some instances, the DNA or RNA is single-stranded. In other instances, the DNA or RNA is double-stranded. In some instances the nuclease is an endonuclease. In still other embodiments, the inhibitor of Shc gene expression is administered to perform RNA interference, antisense therapy, CRISPR genome editing (including CRISPR genome regulation and CRISPR interference), a virus-mediated knockdown, or a combination thereof.

In other aspects, the methods of the present invention for preventing or treating a fibrotic disease in a subject comprise administering to the subject an effective amount of a peptide inhibitor of Shc, a peptoid inhibitor of Shc, a peptide-peptoid hybrid inhibitor of Shc, or a combination thereof to achieve pharmacological suppression of Shc protein activity in the subject.

In some embodiments, the fibrotic disease is selected from the group consisting of fibrotic liver disease, pulmonary fibrosis, cardiac fibrosis, and cystic fibrosis. In particular embodiments, the fibrotic disease is a fatty liver disease. In some instances, the fatty liver disease is non-alcoholic fatty liver disease (NAFLD). In other instances, the fatty liver disease is non-alcoholic steatohepatitis. In yet other embodiments, the subject has a plurality of fibrotic diseases or is at risk for developing one or more fibrotic diseases. In some instances, the subject has diabetes or has one or more risk factors for developing diabetes. In other instances, the subject is obese or has one or more risk factors for becoming obese.

In some embodiments, a peptide inhibitor is used to achieve pharmacological suppression of Shc protein activity. In some instances, the peptide inhibitor is a peptide comprising the sequence pY-Q-G-L-S-amide (SEQ ID NO:4), NH—(CH₂)₂—CO-pY-Q-G-L-S-amide (SEQ ID NO:5), NH—(CH₂)₃—CO-pY-Q-G-L-S-amide (SEQ ID NO:6), NH—(CH₂)₄—CO-pY-Q-G-L-S-amide (SEQ ID NO:7), NH—(CH₂)₅—CO-pY-Q-G-L-S-amide (SEQ ID NO:8), NH—(CH₂)₂—CO-L-pY-Q-G-L-S-amide (SEQ ID NO:9), NH—(CH₂)₃—CO-L-pY-Q-G-L-S-amide (SEQ ID NO:10), NH—(CH₂)₄—CO-L-pY-Q-G-L-S-amide (SEQ ID NO:11), or a combination thereof, wherein “pY” represents phosphorylated tyrosine, and “L,” “Q,” “G,” and “S” are standard one-letter amino acid symbols. In other instances, the peptide inhibitor is a peptide as shown in FIG. 4A, FIG. 4B, or a combination thereof.

In other embodiments, a peptoid inhibitor is used to achieve pharmacological suppression of Shc protein activity.

In still other embodiments, a peptide-peptoid hybrid inhibitor is used to achieve pharmacological suppression of Shc protein activity. In some instances, the peptide-peptoid hybrid inhibitor is a peptide shown in FIG. 4A or 4B, wherein the nitrogen atom depicted at position #2 has been N-allyl substituted. In other instances, the peptide-peptoid inhibitor is the compound shown in FIG. 4C, wherein the “R” group is selected from the group consisting of ethyl, n-propyl, n-butyl, n-pentyl, n-hexyl, 3-methylbutyl, 4-methylpentyl, 4-fluorobutyl, 4,4,4-trifluorobutyl, 2-(methoxy)ethyl, 2-(ethoxy)ethyl, 2-(ethylthio)ethyl, allyl, but-3-en-1-yl, pent-4-en-1-yl, hex-5-en-1-yl, but-2-yn-1yl, pent-2-yn-1-yl, and benzyl. In yet other instances, the “R” group is an alkyl group selected from the group consisting of halo, hydroxy, amino, alkylamino, amido, acyl, nitro, cyano, and alkoxy.

In still other aspects, the methods of the present invention for preventing or treating a fibrotic disease in a subject comprise administering to the subject an effective amount of an inhibitor of Shc gene expression to achieve genetic suppression of Shc activity in the subject and an effective amount of a peptide inhibitor of Shc, a peptoid inhibitor of Shc, a peptide-peptoid hybrid inhibitor of Shc, or a combination thereof to achieve pharmacological suppression of Shc protein activity in the subject. The inhibitor of Shc gene expression and the peptide inhibitor, peptoid inhibitor, peptide-peptoid hybrid inhibitor, or combination thereof may be co-administered or administered separately.

In some embodiments, the isoform of Shc that is inhibited is selected from the group consisting of p46Shc, p52Shc, p66Shc, and a combination thereof. In other embodiments suppression of Shc activity comprises modulating the phosphorylation of tyrosine residues 239, 240, and/or 318 of p52Shc.

In other embodiments, the Shc inhibitor (e.g., inhibitor of Shc gene expression or peptide, peptoid or peptide-peptoid hybrid inhibitor of Shc protein activity) is administered before the subject exhibits any symptoms of a fibrotic disease. In still other embodiments, the subject has one more symptoms of a fibrotic disease (i.e., before the Shc inhibitor is administered). In some instances the subject has received a diagnosis of a fibrotic disease based upon the one or more symptoms before the Shc inhibitor is administered. In yet other embodiments, the administration of the Shc inhibitor ameliorates at least one of the one or more symptoms.

In some embodiments, the level of one or more biomarkers indicative of fibrotic disease in a sample obtained from the subject is abnormal. In some instances, the one or more biomarkers of fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ). In still other embodiments, the level of one or more biomarkers indicative of liver disease in a sample obtained from the subject is abnormal. In some instances, the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin. In particular instances, the ratio of AST to ALT is greater than 2. In particular instances, the sample is serum, plasma, blood, or tissue obtained from a biopsy.

In other embodiments, an imaging technique (e.g., magnetic resonance elastography (MRE), magnetic resonance imaging (MRI), computed tomography (CT) or another X-ray imaging technique, or ultrasonography) is used to assess the presence or extent of fibrosis and/or the response to treatment. In some instances, the imaging technique is used in conjunction with a laboratory test (e.g., a measurement of one or more biomarkers in a blood test), a tissue biopsy, or a combination thereof.

In some embodiments, the Shc inhibitor (e.g., inhibitor of Shc gene expression or peptide, peptoid or peptide-peptoid hybrid inhibitor of Shc protein activity) is administered prophylactically before any biomarkers have abnormal values. In some other embodiments, the levels of one or more biomarkers are abnormal before the Shc inhibitor is administered. In some instances, the subject has received a diagnosis of a fibrotic disease based upon the one or more abnormal biomarker levels. In other instances, the diagnosis is also based upon one or more imaging studies and/or a biopsy. In particular instances, the diagnosis is made before administration of the Shc inhibitor begins.

In other embodiments, administration of the Shc inhibitor (e.g., inhibitor of Shc gene expression or peptide, peptoid or peptide-peptoid hybrid inhibitor of Shc protein activity) results in the level of at least one of the one or more biomarkers returning to a control level. In some instances, the control level is a value that was measured in the subject before treatment with the Shc inhibitor began. In other instances, the control level is a value that was measured in the subject before the subject exhibited any symptoms of the fibrotic disease. In yet other instances, the control level is a value that is obtained from one or more individuals who have not had the fibrotic disease and/or are not at risk for developing the fibrotic disease. In other instances the control level is determined from one or more individuals who were previously successfully treated for the fibrotic disease. In still other embodiments, successful treatment with the Shc inhibitor is evidenced by an imaging study and/or a follow up biopsy.

In particular embodiments, the one or more symptoms are ameliorated after one or more (e.g., 1, 2, 3, 4, 5, 6, 7, 8, 9, 10, or more) dosages of the Shc inhibitor (e.g., inhibitor of Shc gene expression or peptide, peptoid or peptide-peptoid hybrid inhibitor of Shc protein activity). In other embodiments, the levels of the one or more biomarkers return to control levels after one or more (e.g., 1, 2, 3, 4, 5, 6, 7, 8, 9, 10, or more) dosages of the Shc inhibitor. In still other embodiments, the inhibition of Shc activity is transient (i.e., the inhibition of Shc activity abates after several days (e.g., 1, 2, 3, 4, 5, 6, or 7 days), several weeks (e.g., 1, 2, 3, 4, 5, 6, 7, 8, 9, 10, 11, 12, 13, or 14 weeks), or several months (e.g., 1, 2, 3, 4, 5, 6, 7, 8, 9, 10, 11, or 12 months)).

In other embodiments, Shc gene expression is reduced by at least about 5, 10, 15, 20, 25, 30, 35, 40, 45, 50, 55, 60, 65, 70, 75, 80, 85, 90, 5, or 100 percent relative to a control value. In still other embodiments, Shc activity is reduced by at least about 5, 10, 15, 20, 25, 30, 35, 40, 45, 50, 55, 60, 65, 70, 75, 80, 85, 90, 5, or 100 percent relative to a control value.

### V. Therapeutic Administration

A. Pharmaceutical Compositions

Compounds of the present invention are useful in the manufacture of a pharmaceutical composition or a medicament. In certain aspects, a pharmaceutical composition or medicament can be administered to a subject for the prevention or treatment of a fibrotic disease, including NAFLD, NASH, and the like.

Pharmaceutical compositions or medicaments for use in the present invention can be formulated by standard techniques using one or more physiologically acceptable carriers or excipients. Suitable pharmaceutical carriers are described in, e.g., “Remington's Pharmaceutical Sciences” by E. W. Martin. Compounds and agents of the present invention and their physiologically acceptable salts and solvates can be formulated for administration by any suitable route, including, but not limited to, orally, topically, nasally, rectally, parenterally (e.g., intravenously, subcutaneously, intramuscularly, etc.), and combinations thereof. In preferred embodiments, the pharmaceutical composition is administered orally. In some embodiments, the therapeutic agent is dissolved in a liquid, for example, water.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient, i.e., a Shc inhibitor (e.g., a peptide, peptoid, or peptide-peptoid hybrid inhibitor), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethylene glycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethylene glycol 6000 and titatium dioxide. Tablets may be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

In some embodiments, the therapeutic agent is prepared with a polysaccharide such as chitosan or derivatives thereof (e.g., chitosan succinate, chitosan phthalate, etc.), pectin and derivatives thereof (e.g., amidated pectin, calcium pectinate, etc.), chondroitin and derivatives thereof (e.g., chondroitin sulfate), and alginates.

In some embodiments, the therapeutic agent provided herein is loaded onto polymeric nanoparticles that can target a desired site of fibrosis or inflammation. Examples of nanoparticles include biodegradable nanoparticles, pH-sensitive nanoparticles (e.g., comprising Eudragit® S100), trimethylchitosan nanoparticles, polymeric nanoparticles (e.g., comprising PLGA, PEG-PLGA and/or PEG-PCL), and mannose-grafted polymeric nanoparticles. See, e.g., Coco et al., Int. J. Pharm., 440:3-12 (2013).

The therapeutic agent can be encapsulated in a controlled drug-delivery system such as a pressure controlled delivery capsule (see, e.g., Takaya et al., J. Control Rel., 50:111-122 (1998)), an osmotic controlled drug delivery system, and the like. The pressure controlled delivery capsule can contain an ethylcellulose membrane. The osmotic controlled drug delivery system can be a single or more osmotic unit encapsulated with a hard gelatin capsule (e.g., capsule osmotic pump; commercially available from, e.g., Alzet, Cupertino, Calif.). Typically, the osmotic unit contains an osmotic push layer and a drug layer, both surrounded by a semipermeable membrane.

The therapeutic agent can also be formulated in rectal compositions, for example, suppositories or retention enemas, for example, containing conventional suppository bases, for example, cocoa butter or other glycerides.

In certain embodiments, the pharmaceutical composition or medicament of the present invention comprises (I) an effective amount of a compound (e.g., one or more Shc inhibitors) as described herein, and optionally (ii) another therapeutic agent. When used with a compound of the present invention, such optional therapeutic agent may be used individually, sequentially, or in combination with one or more other such therapeutic agents (e.g., a first therapeutic agent, a second therapeutic agent, and a compound of the present invention). Administration may be by the same or different route of administration or together in the same pharmaceutical formulation.

B. Dosage

Pharmaceutical compositions or medicaments can be administered to a subject at a therapeutically effective dose to prevent, treat, reduce, or control fibrosis, fatty liver disease, NAFLD, NASH, and the like, as described herein. In some embodiments, the pharmaceutical composition or medicament is administered to a subject in an amount sufficient to elicit an effective therapeutic response in the subject.

The dosage of active agents administered can be dependent on the subject's body weight, age, individual condition, surface area or volume of the area to be treated and on the form of administration. The size of the dose can also be determined by the existence, nature, and extent of any adverse effects that accompany the administration of a particular compound in a particular subject. Typically, a dosage of the active compounds of the present invention is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

In some embodiments, the therapeutic agent is administered one or more times a day, e.g., 1, 2, 3, 4, 5, 6, 7, 8, 9, 10, or more times a day.

In some embodiments, the therapeutic agent is administered for about 1 to about 31 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the therapeutic agent is administered for at least 1 day. In other embodiments, the therapeutic agent is administered for one or more weeks, e.g., 1, 2, 3, 4, 5, 6, 7, 8, 9, 10, 11, 12, 13, 14, or more weeks. In yet other embodiments, the therapeutic agent is administered for one or more months, e.g., 1, 2, 3, 4, 5, 6, 7, 8, 9, 10, 11, 12, or more months.

To achieve the desired therapeutic effect, compounds or agents may be administered for multiple days at the therapeutically effective daily dose. Thus, therapeutically effective administration of compounds to treat a pertinent condition or disease described herein in a subject may require periodic (e.g., daily or twice daily) administration that continues for a period ranging from three days to two weeks or longer. While consecutive daily doses are a possible route to achieve a therapeutically effective dose, a therapeutically beneficial effect can also be achieved if the agents are not administered daily. For example, one can administer the agents every day, every other day, or, if higher dose ranges are employed and tolerated by the subject, twice a week.

Optimum dosages, toxicity, and therapeutic efficacy of such compounds or agents may vary depending on the relative potency of individual compounds or agents and can be determined by standard pharmaceutical procedures in experimental animals, for example, by determining the LDso (the dose lethal to 50% of the population) and the EDso (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

The data obtained from, for example, animal studies can be used to formulate a dosage range for use in humans. The dosage of such compounds lies preferably within a range of circulating concentrations that include the EDso with little or no toxicity. The dosage can vary within this range depending upon the dosage form employed and the route of administration.

A dose can be formulated in animal models to achieve a concentration range that includes the ICso (the concentration of the agent that achieves a half-maximal inhibition of symptoms) as determined in cell culture. Such information can be used to more accurately determine useful doses in humans. Levels in blood or a tissue sample can be measured, for example, by high performance liquid chromatography (HPLC). In general, the dose equivalent of agents is from about 1 ng/kg to about 100 mg/kg for a typical subject.

The dosage of a pharmaceutical composition of the present invention can be monitored and adjusted throughout treatment, depending on severity of symptoms, frequency of recurrence, and/or the physiological response to the therapeutic regimen. Those of skill in the art commonly engage in such adjustments in therapeutic regimens.

### VI. Monitoring Efficacy

Clinical efficacy can be monitored by measuring one or more of the disease parameters or physical symptoms of fibrotic disease, including but not limited to blood glucose levels, triglyceride levels, blood clotting ability, high body mass index, body weight, blood pressure, blood oxygen levels, heart rate, edema, and skin color. Observation of the stabilization, improvement and/or reversal of one or more symptoms indicates that the treatment or prevention regime is efficacious. Observation of the progression, increase or exacerbation of one or more symptoms indicates that the treatment or prevention regime is not efficacious.

Clinical efficacy can also be monitored using biomarkers. Biomarkers for assessing treatment are preferably assessed at the protein level, but measurement of other biomarkers such as nucleic acids can also be used as a surrogate measure of biomarker expression. Such a level can be measured in a blood sample or in a tissue sample (e.g., biopsied tissue). The level of some biomarkers are reduced in subjects with impaired hepatic function, renal function, cardiac function, pulmonary function, obesity, diabetes, or other sequelae of fibrotic disease relative to a control population of undiseased individuals. An increase in level of such a marker provides an indication of a favorable treatment response, whereas an unchanged or decreasing level provides an indication of unfavorable or at least non-optimal treatment response. The level of other biomarkers is increased in subjects with impaired hepatic function, renal function, cardiac function, pulmonary function, obesity, diabetes, or other sequelae of fibrotic disease relative to a control population of undiseased individuals. A decrease in level of such a biomarker provides an indication of a favorable treatment response, whereas an unchanged or increasing level provides an indication of unfavorable or at least non-optimal treatment response. Non-limiting examples of biomarkers that are useful for the assessment of fibrotic disease are alpha-smooth muscle actin (αSMA), procollagen α1 (procol1), transforming growth factor-β (TGFβ), monocyte chemoattractant protein-1 (MCP1), interleukin-1β(IL-1b), tumor necrosis factor alpha (TNFα), connective tissue growth factor (CTGF), and platelet derived growth factor receptor beta (PDGFRβ). Non-limiting examples of biomarkers that are useful for the assessment of liver disease are aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin.

Also useful for monitoring efficacy are various imaging techniques. Non-limiting examples of useful imaging techniques are magnetic resonance elastography, magnetic resonance imaging, computed tomography and other X-ray imaging techniques, and ultrasonography.

The monitoring methods can entail determining baseline values of one or more measurable biomarkers or disease parameters in a subject before administering a dosage of the one or more Shc inhibitors described herein, and comparing this with a value for the same measurable biomarkers or parameters after a course of treatment.

In other methods, a control value (which may include a mean and standard deviation) of one or more measurable biomarkers or parameters is determined for a control population. In some methods, a control population is one in which subjects have not received prior treatment (e.g., genetic or pharmacological Shc inhibition) and do not have a target disease, nor are at known risk of developing a target disease. In such methods, if the value of the measurable biomarker or clinical parameter approaches the control value, then treatment is considered efficacious. In other methods, the individuals in the control population have not received prior treatment (e.g., genetic or pharmacological Shc inhibition) and have been diagnosed with a target disease. In such methods, if the value of the measurable biomarker or clinical parameter approaches the control values, then treatment is considered inefficacious.

In other methods, a subject who is not presently receiving treatment (e.g., genetic or pharmacological Shc inhibition) but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation or preferably two standard deviations) determined in a population of subjects after undergoing a course of treatment. Alternatively, the measured value in the subject can be compared with a control value in a population of prophylactically treated (e.g., genetic or pharmacological Shc inhibition) subjects who remain free of symptoms of disease, or a population of therapeutically treated subjects who show amelioration of disease characteristics. In such cases, if the value of the measurable biomarker or clinical parameter approaches the control value, then treatment is considered efficacious and need not be resumed. On the other hand, a significant difference relative to the control level (i.e., more than one or preferably two standard deviations) is an indicator that treatment should be resumed in the subject.

### VII. EXAMPLE

The present invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### Example 1

### Shc Knock-Down Ameliorates Fatty Liver Disease

This example shows that both genetic and pharmacological inhibition of Shc activity provide protection from fatty liver disease in vivo and in vitro.

### Introduction

Fatty liver diseases, including non-alcoholic fatty liver disease (NAFLD) and non-alcoholic steatohepatitis (NASH) are serious, life-threatening diseases that worsen with obesity and poor diet, and for which the only long-term solution currently available is liver transplantation. As transplantation is associated with high risk and cost, the identification of genetic and pharmacological means to suppress fatty liver disease are an appealing solution. Genetic Shc depletion in animals has been shown to improve insulin sensitivity and oppose obesity (Tomilov, et al., Aging Cell, 10:55-65 (2011); Tomilov, et al., Aging Cell, 13:1049-1058 (2014); Tomilov, et al., J. Biol. Chem., 291:12575-12585 (2016)), while pharmacological Shc inhibitors are known to increase insulin sensitivity and cellular fat oxidation.

### Results

1. The “fatty liver diet” increases Shc protein quantity and activity.

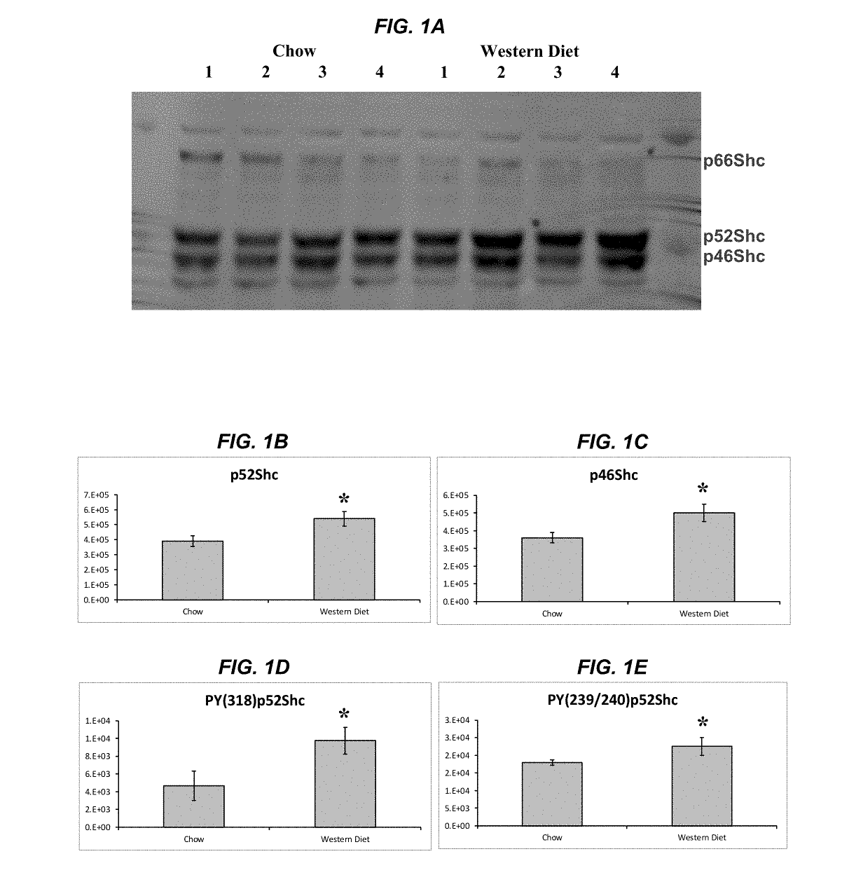

Shc expression was measured under conditions in mice that are known to produce fatty liver disease, i.e., feeding of a high-fat diet (also known as a “NASH” diet or western diet (WD)). Mice were fed either normal chow (n=4) or the WD (n=4) for 20 weeks. Expression of the Shc isoforms p46Shc and p52Shc were significantly increased in mice that were fed the WD as compared to mice that were fed normal chow (FIGS. 1A-1C; P<0.05 for FIGS. 1B and 1C). Furthermore, phosphorylation at tyrosine residues 318 of p52Shc and 239/240 of p52 Shc, which are known to trigger Shc activation, was significantly increased in mice that were fed the WD as compared to mice that were fed normal chow (FIGS. 1D-1E; P<0.05 for both). Thus, a fatty-liver promoting western diet increased the quantity and activity of Shc.

2. Genetic and pharmacological Shc inhibition suppresses fat-induced toxicity in mouse hepatocytes.

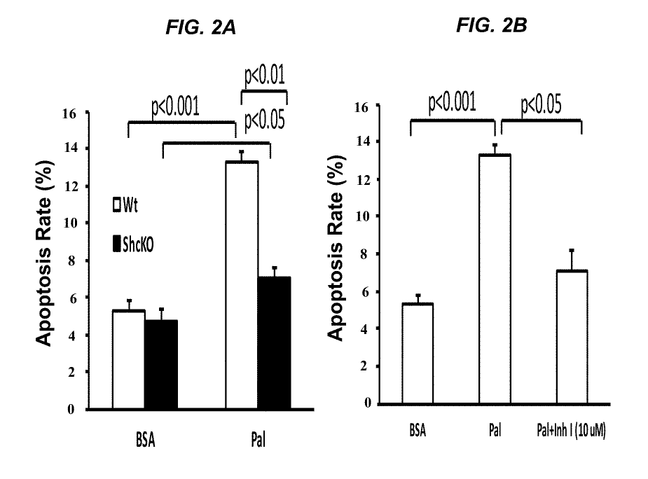

In the standard in vitro assay of fatty liver disease, hepatocytes are presented with 100 uM palmitate (a lipid), and tested for cell death 24 hours later. Hepatocytes from wild type control mice and Shc-depleted mice (ShcKO) were tested for their sensitivity to palmitate using the standard assay. As shown in FIG. 2A, hepatocytes from control mice that were exposed to 100 uM palmitate for 24 hours showed the standard sensitivity to palmitate toxicity (i.e., a nearly three-fold increase in the cell death rate (compare white bars for BSA and palmitate groups in FIG. 2A) (P<0.001)). By contrast, hepatocytes from mice with Shc depletion were very strongly protected from palmitate toxicity (FIG. 2A, black bars). As shown in FIG. 2A (white bars) and FIG. 2B, hepatocytes from control mice that were treated with 100 uM palmitate exhibited the standard sensitivity to palmitate toxicity (P<0.001), while hepatocytes that were co-incubated with palmitate and the Shc inhibitor idebenone were protected from palmitate-induced cell death, showing a nearly 50 percent reduction in apoptosis rate compared to hepatocytes incubated with palmitate alone (P<0.05). Thus both genetic depletion of Shc and pharmacological inhibition of Shc provide protection from lipid-induced hepatotoxicity in vitro.

3. Genetic Shc knock-down ameliorates fatty liver disease after 19 weeks of in vivo non-alcoholic fatty liver disease (NAFLD) development.

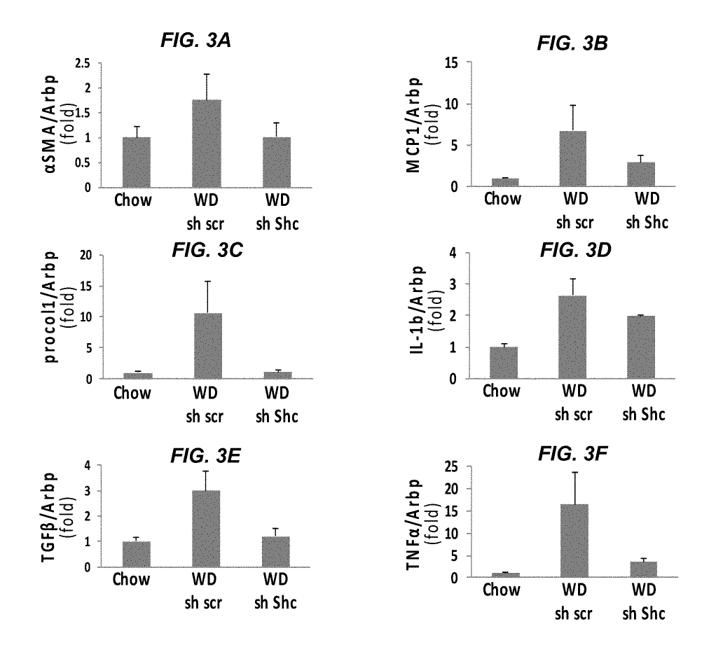

A standard in vivo assay for fatty liver/non-alcoholic fatty liver disease (NAFLD)/non-alcoholic steatohepatitis (NASH) includes a 20-week exposure of mice to a high-fat western diet (WD), after which time they exhibit a large increase in the expression of inflammatory markers of liver disease, including those listed in FIG. 3. Mice were fed normal chow or a WD for 20 weeks. At the end of the 19th week, either a Shc knockdown construct (sh Shc) or a scrambled shRNA construct that is not specific for Shc (sh scr) was injected into the tail veins of the WD-fed mice. Tail vein injections of the constructs occurred twice during the 20^{th} week. At the end of the 20th week, the mice were sacrificed, tissues were harvested, and the expression levels of several markers of liver inflammation were determined, including alpha-smooth muscle actin (αSMA), procollagen al (procol1), transforming growth factor-β (TGFβ ), monocyte chemoattractant protein-1 (MCP1), interleukin-1β (IL-1b), and tumor necrosis factor alpha (TNFα). Every inflammatory marker of liver disease was increased as a result of the fatty WD and every marker was suppressed by Shc inhibition (FIGS. 3A-3F).

### Summary

Both genetic and pharmacological inhibition of Shc activity provide protection from fatty liver disease in vivo and in vitro as shown above. These data show that genetic suppression of Shc (through gene therapies, oligonucleotides, or other knockdown constructs) and pharmacological inhibition of Shc activity are novel and cost-effective therapeutic strategies for life-threating fatty liver disease.

### VIII. Exemplary Embodiments

Exemplary embodiments provided in accordance with the presently disclosed subject matter include, but are not limited to, the claims and the following embodiments:

1. A method for preventing or treating a fibrotic disease in a subject, the method comprising administering to the subject an effective amount of an inhibitor of Shc gene expression to achieve genetic suppression of Shc activity in the subject.

2. The method of embodiment 1, wherein the fibrotic disease is selected from the group consisting of fibrotic liver disease, pulmonary fibrosis, cardiac fibrosis, and cystic fibrosis.

3. The method of embodiment 1 or 2, wherein the inhibitor of Shc gene expression comprises DNA, RNA, a nuclease, or a combination thereof

4. The method of any one of embodiments 1 to 3, wherein the inhibitor of Shc gene expression is administered to perform RNA interference, antisense therapy, CRISPR genome editing, a virus-mediated knockdown, or a combination thereof. 5. The method of any one of embodiments 1 to 4, wherein the inhibitor of Shc gene expression is administered before the subject exhibits any symptoms of the fibrotic disease.

6. The method of any one of embodiments 1 to 4, wherein the subject exhibits one or more symptoms of the fibrotic disease.

7. The method of embodiment 6, wherein the administration of the inhibitor of Shc gene expression ameliorates at least one of the one or more symptoms.

8. The method of any one of embodiments 1 to 7, wherein the suppression of Shc activity in the subject is transient.

9. The method of any one of embodiments 1 to 8, wherein the level of one or more biomarkers indicative of the fibrotic disease is abnormal.

10. The method of embodiment 9, wherein the one or more biomarkers indicative of the fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ).

11. The method of any one of embodiments 1 to 10, wherein the level of one or more biomarkers indicative of liver disease is abnormal.

12. The method of embodiment 11, wherein the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin.

13. The method of any one of embodiments 9 to 12, wherein the level of the one or more biomarkers is measured before administration of the inhibitor of Shc gene expression.

14. The method of any one of embodiments 9 to 13, wherein administration of the inhibitor of Shc gene expression results in the level of at least one of the one or more biomarkers returning to a control level.

15. A method for preventing or treating a fibrotic disease in a subject, the method comprising administering to the subject an effective amount of a peptide inhibitor of Shc, a peptoid inhibitor of Shc, a peptide-peptoid hybrid inhibitor of Shc, or a combination thereof to achieve pharmacological suppression of Shc protein activity in the subject.

16. The method of embodiment 15, wherein the fibrotic disease is selected from the group consisting of fibrotic liver disease, pulmonary fibrosis, cardiac fibrosis, and cystic fibrosis.

17. The method of embodiment 15 or 16, wherein the peptide inhibitor, peptoid inhibitor, peptide-peptoid hybrid inhibitor, or combination thereof is administered before the subject exhibits any symptoms of the fibrotic disease.

18. The method of embodiment 15 or 16, wherein the subject exhibits one or more symptoms of the fibrotic disease.

19. The method of embodiment 18, wherein the administration of the peptide inhibitor, peptoid inhibitor, peptide-peptoid hybrid inhibitor, or combination thereof ameliorates at least one of the one or more symptoms.

20. The method of any one of embodiments 15 to 19, wherein the level of one or more biomarkers indicative of the fibrotic disease is abnormal.

21. The method of embodiment 20, wherein the one or more biomarkers of the fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β (IL-1b), tumor necrosis factor alpha (TNFα), connective tissue growth factor (CTGF), and platelet derived growth factor receptor beta (PDGFRβ).

22. The method of any one of embodiments 15 to 21, wherein the level of one or more biomarkers indicative of liver disease is abnormal.

23. The method of embodiment 22, wherein the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin.

24. The method of any one of embodiments 20 to 23, wherein the level of the one or more biomarkers is measured before administration of the peptide inhibitor, peptoid inhibitor, peptide-peptoid hybrid inhibitor, or combination thereof.

25. The method of any one of embodiments 20 to 24, wherein the administration of the peptide inhibitor, peptoid inhibitor, peptide-peptoid hybrid inhibitor, or combination thereof results in the level of at least one of the one or more biomarkers returning to a control level.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

Informal Sequence Listing

| SEQ ID | | |
|---|---|---|
| NO: | Sequence | Description |
| 1 | Ac-pY-Q-G-L-S-NHR | Peptide/peptoid hybrid |
| | | precursor |
| 2 | Ac-pY-Q-G-L-NHR | Peptide/peptoid hybrid |
| | | precursor |
| 3 | Ac-pY-Q-G-L-amide | Peptide/peptoid hybrid |
| | | precursor |
| 4 | pY-Q-G-L-S-amide | Shc SH2 domain inhibitor |
| 5 | NH—(CH₂)₂—CO-pY- | Shc SH2 domain inhibitor |
| | Q-G-L-S-amide | |
| 6 | NH—(CH₂)₃—CO-pY- | Shc SH2 domain inhibitor |
| | Q-G-L-S-amide | |
| 7 | NH—(CH₂)₄—CO-pY- | Shc SH2 domain inhibitor |
| | Q-G-L-S-amide | |
| 8 | NH—(CH₂)₅—CO-pY- | Shc SH2 domain inhibitor |
| | Q-G-L-S-amide | |
| 9 | NH—(CH₂)₂—CO-L-pY- | Shc SH2 domain inhibitor |
| | Q-G-L-S-amide | |
| 10 | NH—(CH₂)₃—CO-L-pY- | Shc SH2 domain inhibitor |
| | Q-G-L-S-amide | |
| 11 | NH—(CH₂)₄—CO-L-pY- | Shc SH2 domain inhibitor |
| | Q-G-L-S-amide | |
| 12 | X-pY-Q-G-L-S-amide | Peptide inhibitor consensus |
| | | sequence |

What is claimed is:

1. A method for preventing or treating a fibrotic disease in a subject, the method comprising administering to the subject an effective amount of an inhibitor of Shc gene expression to achieve genetic suppression of Shc activity in the subject.

2. The method of claim 1, wherein the fibrotic disease is selected from the group consisting of fibrotic liver disease, pulmonary fibrosis, cardiac fibrosis, and cystic fibrosis.

3. The method of claim 1, wherein the inhibitor of Shc gene expression comprises DNA, RNA, a nuclease, or a combination thereof.

4. The method of claim 1, wherein the inhibitor of Shc gene expression is administered to perform RNA interference, antisense therapy, CRISPR genome editing, a virus-mediated knockdown, or a combination thereof.

5. The method of claim 1, wherein the inhibitor of Shc gene expression is administered before the subject exhibits any symptoms of the fibrotic disease.

6. The method of claim 1, wherein the subject exhibits one or more symptoms of the fibrotic disease.

7. The method of claim 6, wherein the administration of the inhibitor of Shc gene expression ameliorates at least one of the one or more symptoms.

8. The method of claim 1, wherein the suppression of Shc activity in the subject is transient.

9. The method of claim 1, wherein the level of one or more biomarkers indicative of the fibrotic disease is abnormal.

10. The method of claim 9, wherein the one or more biomarkers indicative of the fibrotic disease is selected from the group consisting of alpha-smooth muscle actin (αSMA), procollagen α1 (procol1), transforming growth factor-β (TGFβ), monocyte chemoattractant protein-1 (MCP1), interleukin-1β(IL-1b), tumor necrosis factor alpha (TNFα), connective tissue growth factor (CTGF), and platelet derived growth factor receptor beta (PDGFRβ).

11. The method of claim 1, wherein the level of one or more biomarkers indicative of liver disease is abnormal.

12. The method of claim 11, wherein the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin.

13. The method of claim 9, wherein the level of the one or more biomarkers is measured before administration of the inhibitor of Shc gene expression.

14. The method of claim 9, wherein administration of the inhibitor of Shc gene expression results in the level of at least one of the one or more biomarkers returning to a control level.

15. A method for preventing or treating a fibrotic disease in a subject, the method comprising administering to the subject an effective amount of a peptide inhibitor of Shc, a peptoid inhibitor of Shc, a peptide-peptoid hybrid inhibitor of Shc, or a combination thereof to achieve pharmacological suppression of Shc protein activity in the subject.

16. The method of claim 15, wherein the fibrotic disease is selected from the group consisting of fibrotic liver disease, pulmonary fibrosis, cardiac fibrosis, and cystic fibrosis.

17. The method of claim 15, wherein the peptide inhibitor, peptoid inhibitor, peptide-peptoid hybrid inhibitor, or combination thereof is administered before the subject exhibits any symptoms of the fibrotic disease.

18. The method of claim 15, wherein the subject exhibits one or more symptoms of the fibrotic disease.

19. The method of claim 18, wherein the administration of the peptide inhibitor, peptoid inhibitor, peptide-peptoid hybrid inhibitor, or combination thereof ameliorates at least one of the one or more symptoms.

20. The method of claim 15, wherein the level of one or more biomarkers indicative of the fibrotic disease is abnormal.

21. The method of claim 20, wherein the one or more biomarkers of the fibrotic disease is selected from the group consisting of alpha-smooth muscle actin (αSMA), procollagen α1 (procol1), transforming growth factor-β (TGFβ), monocyte chemoattractant protein-1 (MCPJ), interleukin-1β (IL-1b), tumor necrosis factor alpha (TNFα), connective tissue growth factor (CTGF), and platelet derived growth factor receptor beta (PDGFRβ).

22. The method of claim 15, wherein the level of one or more biomarkers indicative of liver disease is abnormal.

23. The method of claim 22, wherein the one or more biomarkers indicative of liver disease is selected from the group consisting of aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, and ferritin.

24. The method of claim 20, wherein the level of the one or more biomarkers is measured before administration of the peptide inhibitor, peptoid inhibitor, peptide-peptoid hybrid inhibitor, or combination thereof.

25. The method of claim 20, wherein the administration of the peptide inhibitor, peptoid inhibitor, peptide-peptoid hybrid inhibitor, or combination thereof results in the level of at least one of the one or more biomarkers returning to a control level.

26. The method of claim 11, wherein the level of the one or more biomarkers is measured before administration of the inhibitor of Shc gene expression.

27. The method of claim 11, wherein administration of the inhibitor of Shc gene expression results in the level of at least one of the one or more biomarkers returning to a control level.

28. The method of claim 22, wherein the level of the one or more biomarkers is measured before administration of the peptide inhibitor, peptoid inhibitor, peptide-peptoid hybrid inhibitor, or combination thereof.

29. The method of claim 22, wherein the administration of the peptide inhibitor, peptoid inhibitor, peptide-peptoid hybrid inhibitor, or combination thereof results in the level of at least one of the one or more biomarkers returning to a control level.


SEQUENCE LISTING (12 sequences)

SEQ ID NO: 1 - protein, 5 aa, Artificial Sequence
  Synthetic Construct
  MISC_FEATURE (1)..(1): Xaa = aceytelated, phosphorylated tyrosine
XQGLS

SEQ ID NO: 2 - protein, 4 aa, Artificial Sequence
  Synthetic Construct
  VARIANT (1)..(1): Xaa = aceytelated, phosphorylated tyrosine
XQGL

SEQ ID NO: 3 - protein, 4 aa, Artificial Sequence
  Synthetic Construct
  VARIANT (1)..(1): Xaa = aceytelated, phosphorylated tyrosine
XQGL

SEQ ID NO: 4 - protein, 5 aa, Artificial Sequence
  Synthetic Construct
  VARIANT (1)..(1): Xaa =  phosphorylated tyrosine
XQGLS

SEQ ID NO: 5 - protein, 5 aa, Artificial Sequence
  Synthetic Construct
  VARIANT (1)..(1): Xaa =  phosphorylated tyrosine
XQGLS

SEQ ID NO: 6 - protein, 5 aa, Artificial Sequence
  Synthetic Construct
  VARIANT (1)..(1): Xaa =  phosphorylated tyrosine
XQGLS

SEQ ID NO: 7 - protein, 5 aa, Artificial Sequence
  Synthetic Construct
  VARIANT (1)..(1): Xaa =  phosphorylated tyrosine
XQGLS

SEQ ID NO: 8 - protein, 5 aa, Artificial Sequence
  Synthetic Construct
  VARIANT (1)..(1): Xaa =  phosphorylated tyrosine
XQGLS

SEQ ID NO: 9 - protein, 6 aa, Artificial Sequence
  Synthetic Construct
  VARIANT (2)..(2): Xaa =  phosphorylated tyrosine
LXQGLS

SEQ ID NO: 10 - protein, 6 aa, Artificial Sequence
  Synthetic Construct
  VARIANT (2)..(2): Xaa =  phosphorylated tyrosine
LXQGLS

SEQ ID NO: 11 - protein, 6 aa, Artificial Sequence
  Synthetic Construct
  VARIANT (2)..(2): Xaa =  phosphorylated tyrosine
LXQGLS

SEQ ID NO: 12 - protein, 5 aa, Artificial Sequence
  Synthetic Construct
  VARIANT (1)..(1): Xaa =  phosphorylated tyrosine
XQGLS